(19) United States
(12) Patent Application Publication
(10) Pub. No.: US 2011/0275141 A1
(43) Pub. Date: Nov. 10, 2011

(54) ANAERBOIC DIGESTER AND A METHOD FOR TREATING SLUDGE IN THE DIGESTOR

(76) Inventors: Uri Sapir, Pardes Hanna (IL); Ofer Dressler, Tivon (IL)

(21) Appl. No.: 13/145,343

(22) PCT Filed: Jan. 20, 2010
(86) PCT No.: PCT/IB10/50252

§ 371 Date: Jul. 20, 2011

(30) Foreign Application Priority Data
Jan. 21, 2009 (IL) 196633

(51) Int. Cl. C12S 99/00; C12M 1/00

(52) U.S. Cl. 435/262.5; 435/289.1

(57) ABSTRACT

A method for treating sludge in an anaerobic digester includes the steps of providing a pit in the ground that is lined with a seal and then forming cells in the pit into which a mixture of vegetal waste and sludge that has optionally undergone a pre-acidification stage is provided. After a sealing cover is placed over the digester, an optional trigger is provided that enhances formation of biogas that is recovered from the digester to be utilized for the production of energy and the digested mixture recovered at the end of the use of the digester is utilized as an organic fertilizer.

### BACKGROUND

The present disclosure relates to an anaerobic digester and in particular to a method for anaerobic digestion of sludge and green waste.

Efficient treatment of waste is a growing concern in for example large municipalities. Processes such as wastewater treatment generate a residual semi-solid leftover sludge material. Other waste streams that commonly occur in municipalities include vegetal waste or alternatively called green waste such as cut grass, shrub and tree trimmings, wood shavings, wood chips, vegetative waste including yard waste or agricultural crop remains.

U.S. Pat. No. 5,269,634, the disclosure of which is incorporated herein by reference, describes methods for the anaerobic digestion of various solid waste feed stocks. In one embodiment, landfill cells are provided into which trucks or front end loaders can enter and exit to deposit and remove municipal waste materials. The landfill cells can be hermetically sealed and a leachate collecting and spraying system and a biogas recovery system are provided.

### SUMMARY

The following embodiment and aspects thereof are described and illustrated in conjunction with systems, tools and methods which are meant to be exemplary and illustrative, not limiting in scope.

In an embodiment is provided a method for treating sludge comprising the steps of: providing a pit in the ground, lining the pit with a seal, forming cells in the pit, forming a mixture of sludge and vegetal waste, depositing the mixture into each cell and providing a sealing cover over each cell to form an anaerobic digester system in each cell, capturing biogas from the cells, and retrieving the digested mixture from each cell.

Optionally, the method comprises in each cell at least two vertically stacked layers of said mixture wherein a sealing cover is provided between adjacent layers.

Optionally, the sealing cover between adjacent vertically stacked layers includes earth from a vicinity of the pit.

Optionally, before forming the mixture the sludge undergoes a pre-acidification step to hinder the bio-gasification of the sludge.

Optionally, the pre-acidification step is at least partially formed by holding the sludge under anaerobic conditions.

Optionally, leachate collected in the cells is re-circulated in the cells to enhance degradation and/or bio-gasification in the cells.

Optionally, a neutralizing alkaline substance is introduced into each cell to neutralize the acidity of the mixture and trigger enhanced degradation and/or bio-gasification of the mixture.

Optionally, the neutralizing alkaline substance is introduced into each cell by it being added to leachate being re-circulated the cell.

Optionally, the volume of vegetal waste in the mixture is at a ratio of between 0.5-2 times the volume of sludge.

Optionally, the digested mixture retrieved from each cell is adapted to be utilized as an organic fertilizer.

In an embodiment there is further provided a method for treating sludge comprising the steps of: providing an anaerobic digester in a pit in the ground, introducing sludge to be treated into a pre-acidification step in order to hinder the bio-gasification of the sludge, forming a mixture of the treated sludge and vegetal waste, depositing the mixture into the digester, and providing a neutralizing alkaline substance to the digester to trigger substantial degradation and/or bio-gasification of the mixture.

In an embodiment there is even further provided an anaerobic digester formed in a pit in the ground, a face of the pit being sealed and the pit comprising a plurality of cells located horizontally one in relation to the other, each cell comprising at least two vertically stacked layers that are separated by a sealing cover and each cell being sealed from above, wherein each layer comprises a mixture of sludge and vegetal waste.

In addition to the exemplary aspects and embodiment described above, further aspects and embodiments will become apparent by reference to the figures and by study of the following detailed descriptions.

### BRIEF DESCRIPTION OF THE FIGURES

Exemplary embodiments are illustrated in referenced figures. It is intended that the embodiments and figures disclosed herein are to be considered illustrative, rather than restrictive. The disclosure, however, both as to organization and method of operation, together with objects, features, and advantages thereof, may best be understood by reference to the following detailed description when read with the accompanying figures, in which:

FIG. 1 shows a plan view of an anaerobic digester in accordance with the present disclosure;

FIG. 2 shows a cross sectional view of the digester taken in plane II-II in FIG. 1;

FIG. 3 shows a cross sectional view of the digester taken in plane III-III in FIG. 1; and

FIG. 4 shows a section of FIG. 2;

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated within the figures to indicate like elements.

### DETAILED DESCRIPTION

Attention is first drawn to FIG. 1 to show an anaerobic digester 10 that is formed in a pit 12 in the ground and is divided into cells 14. The pit 12 may be a pre-existing pit in the ground such as an old quarry or mine or it may be dug out intentionally from the ground for the purpose of accommodating the digester 10. Adjacent the digester 10 is a pile of vegetal waste 16 and an optional acidification station 18 is also located at a vicinity of the digester 10.

When first forming the digester 10, the pit 12 is empty of cells 14 and the cells 14 in this example, numbered 1 to 24, are formed in sequence in the pit 12 as will be described herein below. It should be noted that directional terms appearing throughout the specification and claims, e.g. “forward”, “rear”, “up”, “down” etc., (and derivatives thereof) are for illustrative purposes only, and are not intended to limit the scope of the appended claims. In addition it is noted that the directional terms “down”, “below” and “lower” (and derivatives thereof) define identical directions.

The digester 10 in accordance with the present disclosure is adapted to treat sludge and vegetal waste. The sludge may originate for example from a wastewater treatment facility and the vegetal waste which may also be referred to as green waste may include substances such as cut grass, shrub and tree trimmings, wood shavings, wood chips, vegetative waste including yard waste or agricultural crop remains etc. In an embodiment, vegetal waste suitable for the digester 10 in accordance with the present disclosure is in a “raw” non-shaved, chipped, ground, crushed or pounded form such as for example wood originating from demolition of buildings or structures or large tree trimmings. Such “raw” vegetal waste may form a firm substrate for vehicles that may pass thereupon when entering the digester 10 as will be mentioned herein below.

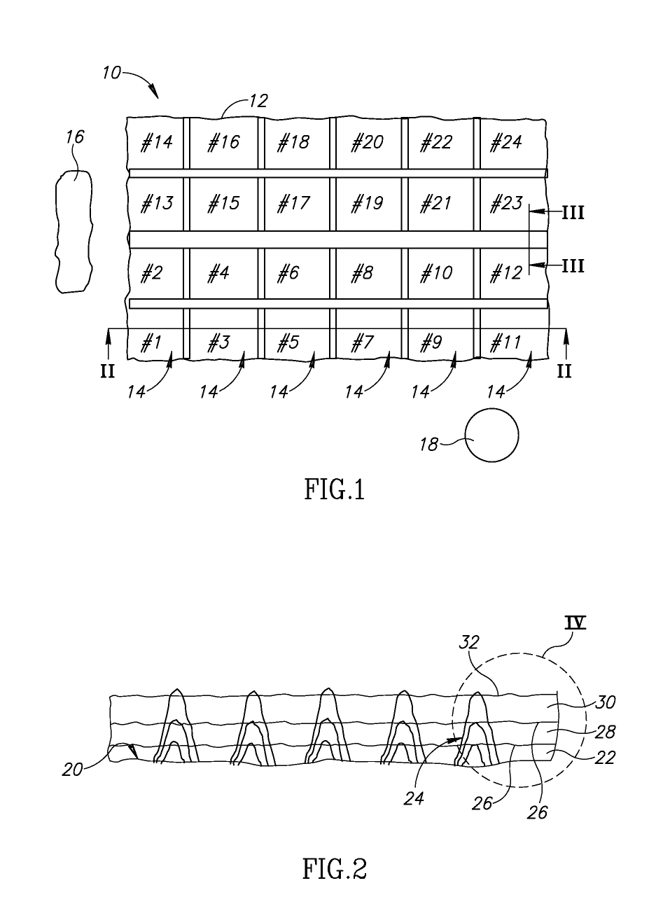

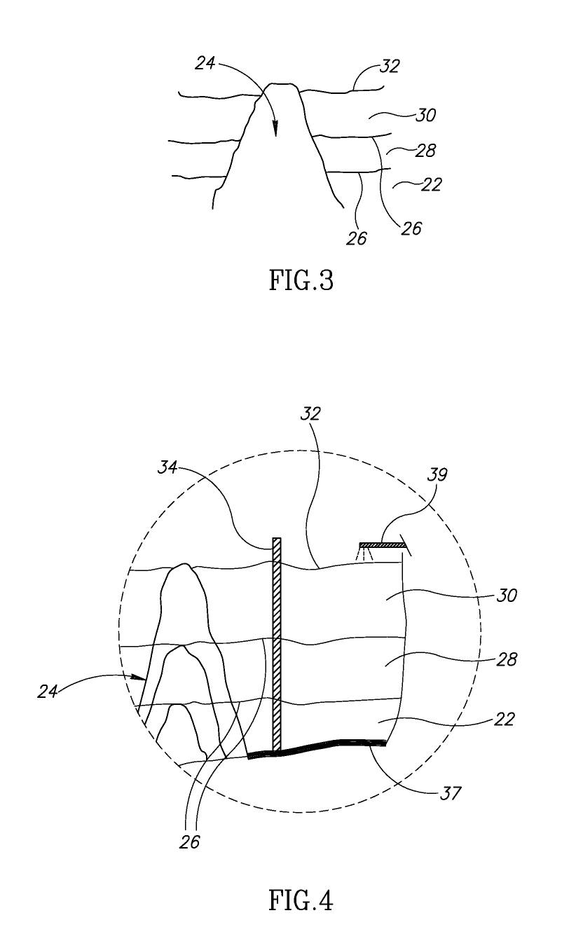

Attention is additionally drawn to FIGS. 2 to 4. Before starting to form the cells 14 in the pit 12, the face 20 of the pit 12 is sealed optionally by placing a polyethylene sheet upon the face 20. The cells 14 as already mentioned above are formed in sequence in the pit 12 with the first cell 14 being formed in this example being cell number 1. Optionally, the first step to forming a cell 14 is to introduce a first layer 22 of sludge and vegetal waste into the cell 14. The inventors have performed practical and theoretical studies of the efficiency of the digester 10 in accordance with the present disclosure. These studies have indicated that optionally the volume of vegetal waste in the mixture should be at a ratio of between 0.5-2 times the volume of sludge and preferably the volume of vegetal waste in the mixture should be substantially equal to that of the sludge.

These studies have taken into consideration the efficiency of the anaerobic digestion process that takes place in the digester 10 and also considerations such as upon what ratio of mixed sludge and vegetal waste can a vehicle traverse when entering a cell to deposit or remove matter to or from the cell 14. A raised strip 24 of optionally earth comprising a first level having for example a height of about 3 meters delimits the first layer 22 in the cell 14. The dimensions of such a first layer 22 may be defined by the volume of mixture that it is possible to deposit in the pit 12 during one working day. When a cell 14 is filled with a first layer 22, the layer 22 is then covered by a sealing cover 26 which is optionally earth that is brought from a vicinity of the pit.

Above the first layer 22, similarly structured second and a third layers 28, may be vertically stacked one on top of the other. For each layer 28, 30 that is added to a cell 14, additional matter is added to the strip 24 that surrounds the cell 14 in order to increase the height of the strip 24 so that it delimits the newly added layer of mixed sludge and vegetal waste. The sealing cover 26 that is optionally formed from earth is provided between adjacent vertically stacked layers 22, 28, 30. Upon the highest layer in a cell 30 a sealing cover 32 is provided that is optionally formed of clay or from a polyethylene sheet. After cell number 1 has been formed in the pit 12, in sequence the remaining cells are then formed one after the other. It is noted that in embodiments of the present disclosure, a cell may have more than three layers. Generally, the number of layers in a cell may in some embodiments be determined by the depth of the pit.

By way of a non binding example, a cell 14 in accordance with an embodiment of the present disclosure has a length of about 40 meters, a width of about 15 meters and a height of about 20 meters.

The vegetal waste that is added to the cells 14 is optionally brought from the pile 16 adjacent the digester 10. The sludge that is added to the cells 14 may be directly transported to each cell 14 from the place where the sludge is created such as the wastewater facility. Optionally, the sludge may be first deposited in an acidification station 18 that is optionally located adjacent the digester 10 before being deposited into the cells 14. In the acidification station 18, the sludge may undergo a pre-acidification stage in order to hinder the bio-gasification of the sludge.

By introducing this step of pre-acidification, the inventors aim to control the timing in which substantial degradation and/or bio-gasification in the cells will start to occur. This may be advantageous for example in order to ensure that the bio-gasification of the mixture will only start to occur after the cells are sealed and ready for use. The pre-acidification stage may optionally be achieved by holding the sludge under anaerobic conditions for a period of 0.5 to 3 days.

In each cell 14, the degradation and/or bio-gasification of the mixed sludge and vegetal waste may cause the release of gases such as methane. These gases may be captured in each cell 14 by a system of collecting pipes 34 that are placed in the cells 14 as shown in FIG. 4. Such a pipe 34 typically includes a perforated lower section and a non-perforated upper section wherein an end of the pipe is coupled to a biogas recovery system (not shown) that recovers the gas (principally biogas) formed in the cells. The lower perforated section is typically located at a lower portion of the cell at a depth that ensures that no air from the outside environment is accidentally sucked into the biogas recovery system. These gases may be utilized for the generation of energy.

In the example where the sludge has undergone a pre-acidification stage, the degradation and/or bio-gasification of the mixture does not substantially start to occur until a certain trigger is provided. Such a trigger may be provided by for example introducing a leachate re-circulating system into the digester. The re-circulating system may optionally be formed by placing a porous layer 37 at a bottom of each cell 14 through which leachate penetrates and then flows by gravitation to a basin (not shown). The leachate is then pumped upwards from the basin to be distributed by a dispenser 39 adjacent the top of each cell 14. This leachate re-circulating system may also be utilized to enhance degradation and/or bio-gasification of sludge that has not undergone a pre-acidification stage.

Another example of a trigger that may be provided in order to enhance the degradation and/or bio-gasification of the mixture may be in the form of a neutralizing alkaline substance that is introduced into each cell 14 to neutralize the acidity of the mixture and trigger enhanced degradation and/or bio-gasification of the mixture. In an embodiment of the present disclosure, the neutralizing alkaline substance may be introduced into each cell 14 by it being added to the leachate being circulated in the cell 14. Another example of a trigger may be in the form of nutrients such as Nitrogen, Phosphorous, iron or other minerals that are added to the re-circulated leachate in the cells 14 in order to enhance the degradation and/or bio-gasification of the mixture.

In the digester 10 in accordance with an embodiment of the present disclosure, the mixed sludge and vegetal waste may be kept in anaerobic digestion conditions in a cell 14 for a relatively long period of time. For example, in an embodiment of the present disclosure the mixed sludge and vegetal waste are kept in a cell 14 for a period of up to two years. During this period of time, the mixed sludge and vegetal waste being digested in each cell 14 are “harvested” for the gases they release for the production of energy. Due to the relatively long period of time that the mixed sludge and vegetal waste remains in the digester 10, the inventors have found that even relatively large tree trimmings that are deposited in the digester are substantially digested at the end of the use of the cell 14. Even if such a large tree trimming is not fully degraded at the end of the use of the cell 14 it can be re-used in a cell that is newly assembled.

At the end of the use of a cell 14, the degraded mixture that is recovered from the cell 14 may be utilized as an organic fertilizer. In an embodiment of the present disclosure it may take about 1 month to create and fill in one cell 14 in the pit 12. Therefore, in the digester 10 shown in FIG. 1 it may take up to two years to form all the 24 cells in the pit. In such a digester 10, when the 24'th cell has been formed in the pit 12 it is possible to start evacuating the 1'st cell in the pit 12 that was formed about two years ago. It is noted that in embodiments of the present disclosure, the mixed sludge and vegetal waste may be kept in a cell 14 for a period of time that longer than two years.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb. Although the present embodiment has been described to a certain degree of particularity, it should be understood that various alterations and modifications could be made without departing from the scope of the disclosure as hereinafter claimed.

1. A method for treating sludge comprising the steps of:
providing a pit in the ground,
lining the pit with a seal,
forming cells in the pit,
forming a mixture of sludge and vegetal waste,
depositing the mixture into each cell and providing a sealing cover over each cell to form an anaerobic digester system in each cell,
capturing biogas from the cells, and
retrieving the digested mixture from each cell.

2. The method according to claim 1 comprising in each cell at least two vertically stacked layers of said mixture wherein a sealing cover is provided between adjacent layers.

3. The method according to claim 2, wherein the sealing cover between adjacent vertically stacked layers includes earth from a vicinity of the pit.

4. The method according to claim 1, wherein before forming the mixture the sludge undergoes a pre-acidification step to hinder the bio-gasification of the sludge.

5. The method according to claim 4, wherein the pre-acidification step is at least partially formed by holding the sludge under anaerobic conditions.

6. The method according to claim 4, wherein leachate collected in the cells is re-circulated in the cells to trigger enhanced degradation and/or bio-gasification in the cells.

7. The method according to claim 4, wherein a neutralizing alkaline substance is introduced into each cell to neutralize the acidity of the mixture and trigger enhanced degradation and/or bio-gasification of the mixture.

8. The method according to claim 7, wherein the neutralizing alkaline substance is introduced into each cell by being added to the leachate being re-circulated in the cell.

9. The method according to claim 1, wherein the volume of vegetal waste in the mixture is at a ratio of between 0.5-2 times the volume of sludge.

10. A method for treating sludge comprising the steps of:
providing an anaerobic digester in a pit in the ground,
introducing sludge to be treated into a pre-acidification step in order to hinder the bio-gasification of the sludge,
forming a mixture of the treated sludge and vegetal waste,
depositing the mixture into the digester, and
providing a neutralizing alkaline substance to the digester to trigger substantial degradation and/or bio-gasification of the mixture.

11. An anaerobic digester formed in a pit in the ground, a face of the pit being sealed and the pit comprising a plurality of cells located horizontally one in relation to the other, each cell comprising at least two vertically stacked layers that are separated by a sealing cover and each cell being sealed from above, wherein each layer comprises a mixture of sludge and vegetal waste.
